(19) United States
(12) Patent Application Publication
(10) Pub. No.: US 2025/0221926 A1
(43) Pub. Date: Jul. 10, 2025

(54) Bioidentical Multimineral Electrolyte BioPolySalt Dispensing Saltshaker

(71) Applicant: Thomas Francis Valone, College Park, MD (US)

(72) Inventor: Thomas Francis Valone, College Park, MD (US)

(73) Assignee: Integrity Research Institute, Beltsville, MD (US)

(21) Appl. No.: 18/406,218

(22) Filed: Jan. 8, 2024

(51) Int. Cl. A61K 9/00; A23L 27/40; A23L 33/16; A61K 33/00; A61K 33/04; A61K 33/06; A61K 33/18; A61K 33/20; A61K 33/24; A61K 33/26; A61K 33/30; A61K 33/32; A61K 33/34; A61K 33/42

(52) U.S. Cl. CPC A61K 9/0056; A23L 27/40; A23L 33/16; A61K 33/00; A61K 33/04; A61K 33/06; A61K 33/18; A61K 33/20; A61K 33/24; A61K 33/26; A61K 33/30; A61K 33/32; A61K 33/34; A61K 33/42

(57) ABSTRACT

The present disclosure relates to a composition of broad-spectrum, multi-mineral mixture which may be packaged in a saltshaker container with through holes at one end. There is a long-standing need for a biocompatible, food grade, polymineral electrolyte salts in a convenient, dispensable shaker container with through-holes to match a similar, if not identical human biological terrain. The object of this invention is to replace the single-mineral sodium chloride dominance of the standard saltshaker with a carefully selected, multi-mineral, bioidentical mixture of electrolyte biopolysalts in granular form with anti-caking agents so the mixed minerals will dispense without clogging or caking up. The purpose is to balance the endogenous electrolyte mix in the body, thus lowering blood pressure, avoiding heart disease, slow and reverse aging, and to counter heat stroke related to internal mineral depletion, in a most convenient, tabletop available manner.

### CROSS REFERENCE TO RELATED APPLICATIONS

This nonprovisional patent application claims the benefit of and priority to U.S. Provisional Application No. 63/438,004 filed on Jan. 10, 2023 by the present inventor.

### BACKGROUND OF THE INVENTION

Salt or specifically, sodium chloride consumption significantly increased to the extent that it became a public-health problem only after WW II, when postwar prosperity bolstered appetites for restaurant meals and presalted, processed and frozen foods. Salt-free cookbooks started appearing in the 1950s and in the 1970s, manufacturers dropped salt from baby foods. (Hereafter, “salt” is used to indicate sodium-based granulated minerals unless otherwise indicated.) By 1981 the FDA launched sodium-education initiatives in the hope of reducing U.S. salt intake. By 2010, sodium was added to the list of ingredients required to be mentioned on nutrition labels and NYC led the National Salt Reduction Initiative to crack down on salt in restaurants and packaged food. However, in spite of government and media awareness efforts, salt consumption has jumped 50% since the 1970s (TIME, Apr. 5, 2010).

In the United States today, 89% of Americans consume twice as much sodium-based salt as they need, causing high blood pressure and premature death (TIME, May 15, 2017), a 26% spike in obesity risk (DrFuhrman.com), linked to a higher death rate (New Scientist, 28 May 2016), and even impair the body's ability to fight bacterial infections as more urea accumulates in the kidneys when salt levels are high (New Scientist, 4 Apr. 2020). Though the multibillion-dollar industry in dietary supplements includes vitamins and minerals and even mineral powder packets thus clearly demonstrating a market demand for such an innovative, universally helpful product, no such multimineral, biocompatible and biophilic polysalt is available for consumers today. Everyone requires some amounts of the essential nutrients in the present disclosure. Supplements are of benefit to anyone within the population even though many people can fulfill their needs with the intake of healthy diets of mixed foods. For those who supplement and those who do not, the use of the sodium chloride saltshaker is almost universal, where even with sea salt or Himalayan salt, the content is one-sided with 98% sodium chloride. New Scientist reports (2 Nov. 2019) that a high-salt diet can “gum up the brain” from an immune response that builds up protein which may stop brain cells from working properly, even among those with normal blood pressure.

Strenuous exercise, especially during athletic workouts can cause the loss of cortisol, amino acids, glucose, carbohydrates, and water, besides creating excessive free radicals. In addition, it is well-known that elderly people can be at risk for dehydration from inadequate fluid intake, since the sense of thirst tends to diminish with age, or from sunstroke, heatstroke, etc. Hypovolemia, the isotonic fluid loss from the extracellular space can result, especially in older patients, which leads to dizziness, confusion, impaired mental state, and in extreme cases, seizure or coma. With global warming setting heat records almost every year now, such hot conditions contribute to thousands of deaths during the summer season.

The American Heart Association, the Department of Health and Human Services, and the Dietary Guidelines Advisory Committee among others have advised that excessively high intake of sodium is very common and widespread with American adults, which can often exceed six grams a day. However, high intake of sodium chloride salt, or in other words, excess sodium due to a high sodium diet increases blood pressure because it holds excess fluid in the body and creates an added burden on the heart. Too much sodium also will increase the risk of stroke, heart failure, osteoporosis, stomach cancer, general immune deficiency, and kidney disease. As a result of such high sodium intake, 1 in 3 Americans will develop high blood pressure in their lifetime and premature death, besides contributing to Alzheimer's disease, according to multiple sources. Therefore, limiting sodium chloride salt intake to 1.5 grams (1,500 milligrams) per day is recommended, especially for those over 50 and for African Americans. Often there are foods that try to compensate for the sodium content by including a small amount of potassium compound. However, all of them (98% or more of them) do not supply enough potassium in the correct 2:1 ratio that would match the body's internal, biological terrain mixture, in order to be biocompatible. Sodium levels at current dietary levels cause biological aging at a faster rate according to a published report (New Scientist, Jan. 2023). Compared to adults with serum sodium levels in the middle of the normal range, those with serum sodium levels at the higher end of the range are more likely to acquire chronic illnesses and exhibit symptoms of advanced biological aging, according to new research. Furthermore, adults with higher levels have an increased risk of passing away earlier in life but the results suggest that proper hydration may slow down aging and prolong a disease-free life. Researchers looked at how serum sodium levels correlated with biological aging, which was assessed through 15 health markers. They also discovered that people even with normal serum sodium levels in the range of 135 to 146 milliequivalents per liter (mEq/L) were more likely to exhibit indications of biological aging at a faster rate. Health practitioners and the instant invention seek to maintain normal serum electrolyte levels.

As a matter of fact, there are many other electrolyte balancing acts occurring in the human body, which are thrown into disarray with such one-sided sodium mineral dietary content that the majority of consumers experience every day exacerbating personal health issues, especially in older people. Another example is calcium, such as in eggshells, which normally needs to be balanced with the proper 2:1 ratio with a magnesium salt for complete absorption. The literature is filled with medical studies that have found a correlation between the intake of unbalanced minerals and their accumulation in the joints, brain, or fatty tissue as mineral deposits which the body cannot digest nor eliminate. These sometimes can be removed by fasting or by chelation therapy administered by a medical or health professional since they can lead to debilitating conditions including arthritis, dementia, etc. However, most people do not use this option in later years when needed the most and often times such chelating agents can and do release arterial plaque into the bloodstream which presents a cardiac risk in the elderly.

Pink Himalayan salt is claimed to be a solution, which is a pink-colored salt extracted from the Khewra Salt Mine located near the Himalayas in Pakistan. Some people estimate it may contain up to 84 different minerals and trace elements. In fact, it's these very minerals, especially iron, that give it its characteristic pink color. The Khewra Salt Mine is one of the oldest and largest salt mines in the world. The pink Himalayan salt harvested from this mine is believed to have been formed millions of years ago from the evaporation of ancient bodies of water. The salt is hand-extracted and minimally processed to yield an unrefined product that's free of additives and thought to be much more natural than table salt. However, just like table salt, pink Himalayan salt is mostly (over 90%) comprised of sodium chloride. However, the natural harvesting process allows pink Himalayan salt to possess many other minerals and trace elements that are not found in regular table salt but only in small albeit tiny amounts. Interestingly, Healthline.com correctly notes that:

“ . . . pink Himalayan salt contains more calcium, potassium, magnesium and iron than table salt. Nevertheless, the amounts of these minerals in pink Himalayan salt are very, very small. They are found in such small quantities that it would take 3.7 pounds (1.7 kg) of pink Himalayan salt to obtain the recommended daily amount of potassium, for instance. Needless to say, that's an unrealistic amount of salt to consume. For the most part, the extra minerals in pink Himalayan salt are found in such small quantities that they are unlikely to provide you with any health benefits whatsoever.”

Multi-mineral dietary citations are in the prior art journal literature such as “Trace minerals in human growth and development”, Castillo-Duran C, Cassorla F., J Pediatr Endocrinol Metab. 1999; 12 (5 Suppl 2): 589-601. http://www.ncbi.nlm.nih.gov/pubmed/10854188. The authors emphasize, “Trace mineral deficiencies may affect several biological functions in humans, including physical growth, psychomotor development and immunity. We have reviewed the mechanisms whereby several trace mineral deficiencies may affect these biological functions at different ages (fetal life, infancy, childhood and adolescence), as well as the evidence supporting this association. We describe the effects of zinc deficiency on the hormonal regulation of growth and sexual development in both humans and animal models. We provide data regarding the effects of iron deficiency on growth and psychomotor development. We mention the effects of copper, manganese, selenium and iodine deficiencies on growth and development. We conclude that iron deficiency may affect psychomotor development but does not appear to affect growth. Zinc deficiency may cause growth retardation and psychomotor delay.” Notably, the authors do not suggest a daily regimen of a wide spectrum of food grade, multi-mineral supplementation with a shaker dispenser but the reader of the article might be motivated to feel the need for such a product.

Of the many publications emphasizing the problems with a dominance of sodium in the diet, many journal articles warn of the dangers, such as “Excess dietary salt intake alters the excitability of central sympathetic networks”, Stocker S D, Madden C J, Sved A F. Physiol Behav. 2010 Jul. 14; 100 (5): 519-24. Epub 2010 May 1. http://www.ncbi.nlm.nih.gov/pubmed/20434471. This article states that

“The ingestion of excess dietary salt (defined as NaCl) is strongly correlated with cardiovascular disease, morbidity, mortality, and is regarded as a major contributing factor to the pathogenesis of hypertension. Although several mechanisms contribute to the adverse consequences of dietary salt intake, accumulating evidence suggests that dietary salt loading produces neurogenically-mediated increases in total peripheral resistance to raise arterial blood pressure (ABP). Evidence from clinical studies and experimental models clearly establishes a hypertensive effect of dietary salt loading in a subset of individuals who are deemed ‘salt-sensitive’. However, we will discuss and present evidence to develop a novel hypothesis to suggest that while chronic increases in dietary salt intake do not elevate mean ABP in ‘non-salt-sensitive’ animals, dietary salt intake does enhance several sympathetic reflexes thereby predisposing these animals and/or individuals to the development of salt-sensitive hypertension. Additional evidence raises an intriguing hypothesis that these enhanced sympathetic reflexes are largely attributed to the ability of excess dietary salt intake to selectively enhance the excitability of sympathetic-regulatory neurons in the rostral ventrolateral medulla. Insight into the cellular mechanisms by which dietary salt intake alters the responsiveness of RVLM circuits will likely provide a foundation for developing new therapeutic approaches to treat salt-sensitive hypertension. The paper represents an invited review by a symposium, award winner or keynote speaker at the Society for the Study of Ingestive Behavior [SSIB] Annual Meeting in Portland, July 2009.”

Jaklenec from Massachusetts Institute of Technology teaches the heat resistant formulations providing particles of micronutrients and vitamins encapsulated in water insoluble polymers (Jaklenec, Ana et al., “Fortified Micronutrient Salt Formulations”, 24 Jun. 2015, WO 2015/095185). The polymers package works on a daily basis to provide recommended doses of vitamins, simultaneously with salt, in order to eliminate the need for daily vitamins. Jaklenec does not teach a dispensable form of a biocompatible, balanced, multi-electrolyte salt mixture.

U.S. Pat. No. 10,576,042 by Vieira teaches Polyelectrolyte Complexes, has a composition of predominantly negatively charge polymer-based polyelectrolyte polymer and a gellan gum. It is designed for tissue engineering and regenerative medicine. it does not offer a granulated, polyelectrolyte mixture of balanced, wide spectrum salts that are biocompatible.

U.S. Pat. No. 9,005,662 by Schlenoff is directed to biocompatible polyelectrolyte complexes that can be considered hydrogels with high crosslinking density, useful for medical and pharmaceutical applications, which is not related to the food grade dispensable form of a polyelectrolyte salt in granulated, saltshaker enclosure of the present invention.

Health Salt, sold by Sundar, is a fortified salt containing a plurality of micronutrients, which is allegedly thermally stable. The formulation appears to be a physical mixture of salt and the various micronutrients. Vinodkumar et al., Int. J. Vitam. Nutr. Res., 79 (5): 348-361 (2009), describes manufacturing a multiple micronutrient-fortified salt in a ribbon blender. Vinodkumar further evaluates only the homogenous distribution of the micronutrients in the salt. However, the product costs 2-3 times more than loose salt and also contains black specks of forticants which were observed in the product. Furthermore, the product has an unusual odor and also, the micronutrients in the product cannot be verified without laboratory testing. Health Salt is notably not taught to replace table salt in a shaker form.

Varun Enterprises, New Delhi, offers a wide range of polyelectrolyte powder designed for water treatment and industrial grade quality. Polyelectrolytes from this source are both flocculants as well as deflocculated depending upon the molecular weight, where the flocculent is essentially a solid liquid separating agent while a deflocculated is a dispersing agent but not intended for a dietary, food grade product.

TABLE 1
Electrolyte Powders on the Market (ref: https://www.garagegymreviews.com)

Electrolyte Powder Overall: Transparent Labs Hydrate V3
Electrolyte Powder for Athletes: Kaged Hydra-Charge
Best-Tasting Electrolyte Powder: XWERKS Motion
On-the-Go Electrolyte Powder: Liquid IV
Low-Sugar Electrolyte Powder: LMNT
Electrolyte Powder for Endurance Athletes: BUBS Naturals Hydrate or Die Packs
Keto Electrolyte Powder: Key Nutrients Electrolyte Recovery Plus
Electrolyte Powder for Everyday Hydration: UCAN Hydrate
Electrolyte Powder for Intra-Workout: Precision Hydration Electrolyte Drink Mix
Electrolyte Powder for Digestion: Pedialyte AdvancedCare Plus Electrolyte Powder
Budget Electrolyte Powder: Propel Electrolyte Water Beverage Mix
Electrolyte Powder for Joint Health: Emergen-C Hydration + Electrolyte Replenishment
Organic Electrolyte Powder: Orgain Hydro Boost
Vegan Electrolyte Powder: Vega Sport Hydrator Electrolyte Powder
Electrolyte Powder With Collagen: Vital Proteins Hydration + Collagen

Table 1 shows an up-to-date comprehensive internet list of the major electrolyte powders on the market today, for an assortment of applications and design purposes. However, they seem to cater only to the fitness enthusiast and not to the general public who never use packets of powder on a regular basis for any reason. Also, once again none of these include the explicit nor implied application suitable for a saltshaker to be used on the tabletop for each meal. Furthermore, Good Housekeeping also evaluated thirty different electrolyte powders to come up with a set of nine electrolyte powders online recommended by dietitians and fitness experts (goodhousekeeping.com/food-products/), thus showing the market demand for electrolytes but an obvious neglect of the general public who are notoriously saltshaker users only and therefore, a wide-open market for a tabletop saltshaker product with similar electrolyte mixture content. Notably, if the instant Biopolysalt shaker invention and its user-friendly, biophilic encapsulation was an obvious variation, the groups of companies mentioned above would have certainly invented it by now.

A known opponent of supplementation which runs counter to even the most blatant sodium overdose of the population is the National Institutes of Health (NIH) that publishes reports such as “Vitamin/mineral supplements: of questionable benefit for the general population,” McCormick D B. Nutr. Rev. 2010 Apr; 68 (4): 207-13, http://www.ncbi.nlm.nih.gov/pubmed/20416017. The NIH states in this report summary that:

“In the United States today, there is a multibillion-dollar industry in dietary supplements with at least a third sold as vitamin/mineral pills and drinks. Though everyone requires small amounts of these essential micronutrients, and supplements are of benefit to some within the population, the considerable majority of people can fulfill their needs with the intake of healthy diets of mixed foods. In addition, the fortification of some processed foods adds extra amounts of several micronutrients, especially those for which there is deemed to be a need in special segments of our population. In spite of this safe and adequate level of intake, there are many who have been led to believe that the frequent ingestion of supplements will be helpful in their efforts to maintain optimal health, live longer, and even prevent or cure non-deficiency diseases. It is the intent of this article to unravel the causes and misconceptions behind this practice and to emphasize that most of the money spent on unnecessary supplementation could be better used for other purposes.”

The NIH is correct in one sense that such ‘healthy diets’ will have a very difficult time to compensate for and neutralize the excessive sodium poisoning of the general population. Therefore, there exists a need for compositions that provide a balanced, multi-mineral salt mixture that matches the biological terrain of the human intercellular fluid so as to be biologically compatible, in a noncaking, saltshaker dispenser for tabletop use, as well as in bulk. Though a few electrolyte salt mixtures are available in capsule form or packet form, none of them are designed, advertised, nor disclosed to be presented in a product form to operate in a saltshaker. An exception a simple potassium chloride monosalt in a shakable form called NuSalt®, both of which aim for a limited market which only addresses a sodium overdose. Nothing on the market today constitutes a multimineral electrolyte or polysalt saltshaker style product.

### SUMMARY OF INVENTION

This system of restorative table salt seeks to revolutionize and replace, in a completely biocompatible manner, the content of the common, mono-mineral saltshaker by proper mixture of electrolyte salts which address the average person's biological need. It specifically does this by dispensing in a container perforated at one end a specified range of mineral salts actually matching, as close as possible, the average inner biological terrain determined by any number of analytically means available to the food research industry, instead of simply providing the sodium-based salt flavor they like for his/her taste. Such a biophilic match of the biological terrain will necessarily include a proper two to one ratio of potassium to sodium, as well as a proper two to one ratio of calcium to magnesium, and a proper biocompatible ratio of 1.6 to one of calcium to phosphorous, with a balance of chloride to bicarbonate. In fact, studies have shown that such a mixture of essential salts like those in the BPS shaker is more flavorful, fulfilling, satisfying and sustaining than the dangerously unhealthy, isolated sodium chloride “monosalt”. The instant invention, a bioidentical multimineral electrolyte BioPolySalt™ (BPS) dispensing saltshaker can be packaged in a cardboard, paper, plastic, or metal encapsulation, enclosure, vial, bottle, or container with perforations in one end that may be the top surface which is reclosable or not, large or small, where the one end may or may not consist of a removable cap or cover which may be snap-on or screw-on style; leaving the packaged product enclosure primarily used as a shakable dispenser, more commonly referred to as a saltshaker, which also can be covered or sealed, that consumers will learn to prefer, instead of the old-fashioned sodium overdose.

### BRIEF DESCRIPTION OF THE DRAWINGS

The present invention is described in reference to the accompanying figures.

FIG. 1 is a graph of the typical distribution and nature of the endogenous electrolytes in a human body. The graph shows the composition of the ICF, IF, and plasma. The compositions of plasma and IF are similar to one another but are quite different from the composition of the ICF.

FIG. 2 is another graph of the compartments distribution of electrolytes in the human where body blood plasma contains a slightly larger total of electrolytes (ions) than interstitial fluids do and blood contains more sodium ions and fewer chloride ions than interstitial fluid does.

FIG. 3 is an illustration of the granules for the BPS mineral salt and a typical shaker that are possible for the present invention, depending on the style and perforated round hole construction of the saltshaker chosen.

### DETAILED DESCRIPTION OF PREFERRED EMBODIMENTS

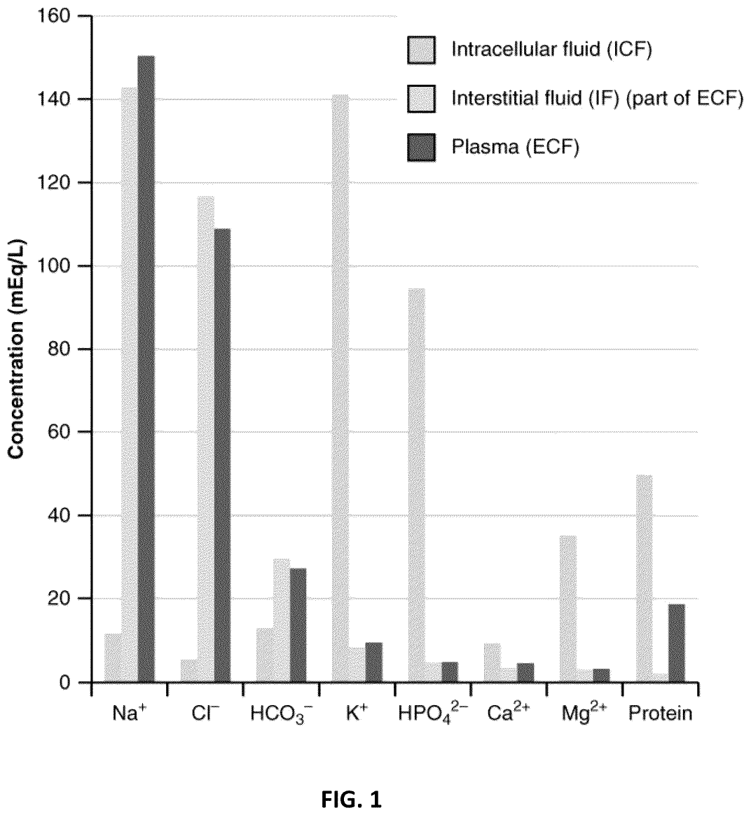

Composition of body fluids consists of the compositions of the two components of the Extra-Cellular Fluid (ECF): blood plasma and interstitial fluid (IF) are more similar to each other than either is to the Intra-Cellular Fluid (ICF) such as shown in FIG. 1. Blood plasma has high concentrations of sodium, chloride, bicarbonate, and protein. The IF has high concentrations of sodium, chloride, and bicarbonate, but a relatively lower concentration of protein. In contrast, the ICF has elevated amounts of potassium, phosphate, magnesium, and protein and is found in the cells (cytoplasm, nucleoplasm) comprises 60% of all body fluids. humor and vitreous body of eyes, between serous and visceral membranes, glomerular filtrate of kidneys. Overall, the ICF contains high concentrations of potassium and phosphate, whereas both plasma and the ECF contain high concentrations of sodium and chloride. (https://courses.lumenlearning.com/suny-ap2/chapter/body-fluids-and-fluid-compartments-no-content/).

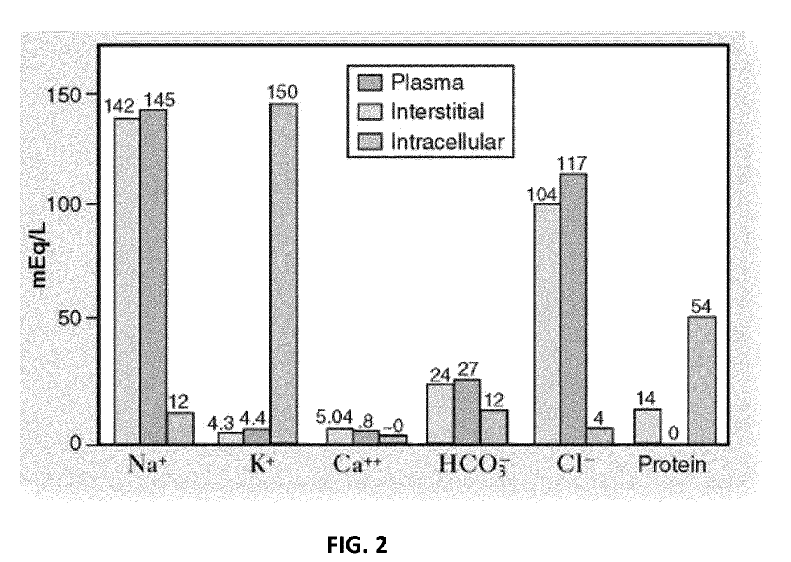

FIG. 2 shows a diagram of compartments and some of the chemical species they contain. H2O, CO2, and Cl—are assumed to have an equilibrium-based distribution among these compartments. Extracellular fluids and intracellular fluid are more unlike than alike chemically. Chemical similarity predominates between the two extracellular fluids. K+ is also assumed to have an equilibrium distribution, but for only three compartments, since it is effectively impermeable to erythrocytes as found by Raftos et al. (Raftos J E, Bulliman B T, Kuchel P W. Evaluation of an electrochemical model of erythrocyte pH using 31P nuclear magnetic resonance data. J Gen Physiol 95:1183-1204, 1990.) This information and compartments differ from that of DeLand and Bradham (DeLand E C, Bradham G B. Fluid balance and electrolyte distribution in the human body. Ann NY Acad Sci 128:795-809, 1966.)

DeLand and Bradham simulated Na+ and K+ distribution across erythrocyte and cellular membranes using a constant, standard-free-energy pump. In contrast, our previously validated IPE model (39) used the erythrocyte model of Raftos et al. who found that besides K+, Na+ was also effectively impermeable to this membrane. Na+ and other small ions not shown, such as Ca2+, Mg2+, Pi-, and lac-, distribute at equilibrium across the I-P microvascular membrane, with the latter two also entering erythrocytes.

This invention may contain as its mixed content, some or all minerals from the complete list of endoscopic, ECF, IF, and ICF electrolytes found in the average human body. A standard sample list is from Medscape.com whose minerals may be represented and optimally compounded with but not limited to a carbonate, bicarbonate, citrate, chloride, etc. for ensured absorbability. The sample list of essential human biological endogenous mineral ingredients for this invention may include the following:

TABLE 2
Magnesium: Critical for bone health, DNA and RNA support, and is responsible for relaxing and
contracting muscles-this is a great tool for combatting PMS and easing digestion and helps carry out
over 300 reactions in the body, including processes related to muscle and nerve function, energy
production, nerve health, and blood sugar control. Whole food sources include quinoa, avocado, spinach,
swiss chard, and pumpkin seeds.
Potassium: Helps with muscle repair and metabolism and maintains proper heart function, regulates
nerve signaling, and controls fluid balance. Food sources include bananas, melon, prunes, and citrus.
Sodium: Found in average table salt, pure sodium helps works in tandem with potassium to regulate the
water in and around your cells-too little, and our cells become inflamed and can cause several health
issues and maintains fluid balance, aids muscle contraction, transmits nerve impulses, and impacts blood
pressure. Whole food sources include celery and beets.
Chloride: In addition to being a critical electrolyte, chloride is an important piece of hydrochloric acid, the
stomach digestive enzyme responsible for breaking down your nutrients and is required for proper
digestion-and eliminating bloat and regulates fluid balance, maintains proper blood pressure, and aids
digestion. Whole food sources include seaweed and other sea vegetables, olives, tomatoes, and lettuce.
Zinc: If supple skin is desirable, zinc is an important tool in achieving the glow, as its powers include
helping with skin elasticity, integrity, and even tone by fighting free radicals, and controlling inflammation.
Whole food sources include chickpeas, lentils, and hemp seeds.
Molybdenum: An important mineral in the detoxification process, the metal activates enzymes that spur
detox within cells and prevents a harmful buildup of sulfites (all the more reason for choosing organic,
natural wines!). Whole food sources include kidney and navy beans, cashews, and tempeh.
Chromium: This trace metal aids in blood sugar stabilization and draws blood sugar out of cells. Whole
food sources include green beans, broccoli, and potatoes.
Calcium: Calcium is critical for bones and helps maintain their integrity-not to mention it is also
responsible for nervous system, heart and muscle vitality as well. Whole food sources include collard
greens, bok choy, and turnip greens.
Phosphorus: Compliments calcium to support proper bone formation and health and regulates nerve and
muscle function, strengthens teeth and bones, grows and repairs tissues, and makes up our genes. Whole
food sources include plants with high protein such as chard, beans, lentils, and nuts.
Silica: Works in tandem with sulfur to detoxify the body and build strong nails and shiny hair. It also
maximizes cellular hydration by making water “wetter”, a phenomenon that creates proper fluid balance
in and around the cells. Dehydrated cells can cause them to stack or cluster, translating to inflammation
and parched skin and organs. Whole food sources can be found in mangoes, spinach, oats and brown rice.
Selenium: This mineral acts as a powerful antioxidant, and aids in thyroid health, hormone balance, and
cognitive function. Just a single Brazil nut provides over 130% of your RDI, but other whole food sources
include shiitake mushrooms and sunflower seeds.
Bicarbonate: maintains the body's acid-base (pH) balance.

However, due to increasing climate global warming with record-breaking, high temperatures in many parts of the world, to ensure the restoration of those electrolyte minerals to the human body under stress and depleted for any reason, the proper restorative biophilic BPS can be utilized instantly in a saltshaker form or in capsule form. This system of restorative dietary table salt includes a proper two to one ratio of potassium to sodium, as well as a proper two to one ratio of calcium to magnesium, and also a properly beneficial biocompatible ratio of 1.6 to one of calcium to phosphorous, with a balance of chloride to bicarbonate. Doctors have seen a rapid recovery from heatstroke by administering electrolytes immediately, thus relieving the delirium and dizziness which accompanies heatstroke, that is the precursor to a fainting spell. In the health literature, such as healthline.com, the deprivation of sufficient minerals is also a health hazard. Therefore, adding sufficient, biocompatible electrolyte mineral salts to the diet via the common saltshaker will be a benefit for the general population as a prophylactic or therapeutic treatment of various deficiencies.

For this biocompatible BPS mixture suitable for a saltshaker which may be made from but not limited to glass, plastic, metal, wood, or ceramic, the list of salts are the essential electrolyte salts taught in this invention but not limited to those electrolytes. The BPS invention may contain a specific amount of those listed in Table 3, to maximize flavor and most importantly to match the endogenous electrolyte content in a similar and preferably, an identical ratio of the electrolyte salts in the human body in a homeostasis condition, along with necessary, state-of-the-art anti-caking additive(s). Such a replenishing, bioidentical match of normal biosalts will also be ideally suitable for replenishing electrolytes lost due to exertion, heat exposure, dehydration, fluid loss, etc. This disclosed system for regulating the contents of the common saltshaker to be biocompatible can very possibly save lives (such as in Qatar where at least 6,500 migrant workers from the cold climate of Nepal have been recruited to work in 110 degree F. temperatures between 2010 and 2020 to work on the World Cup stadium, which caused many to succumb to heatstroke and a huge number estimated to be in the thousands did not survive. Ref: Hauck and Hagen, USA Today, Nov. 30, 2022).

The following compounds of the Table 3 minerals are the suggested list that may be included in the contents of the BPS mixture of the present invention, in a proper biocompatible ratio, designed for anti-caking saltshaker dispensing:

TABLE 3

Potassium citrate
Calcium citrate
Sodium chloride
Magnesium citrate
Zinc ascorbate
Calcium panothenate
Manganese gluconate
Sodium selenite
Chromium picolinate and chromium polypicolinate
Copper citrate
Selenium rice chelate
Potassium Iodide (as granular kelp)
Other trace mineral compounds

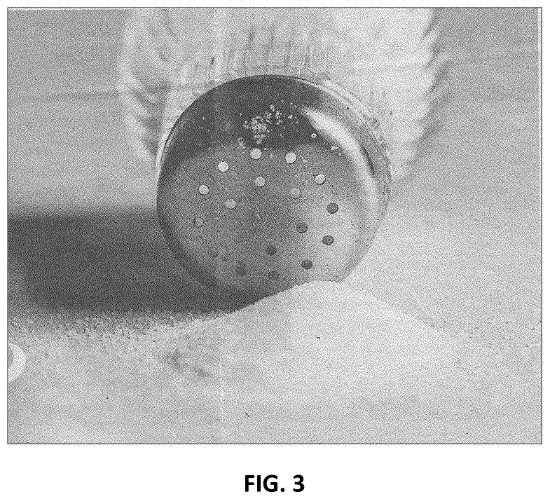

FIG. 3 alludes to the challenges involved in grinding, filtering, possibly with a screen or a sieve, and evaporating to create a fine grain size presented visually, which will normally result in the midrange granules which will pass through the round hole perforated openings that the BPS shaker possesses at one end, being a snap-on or screw-on cap, termination, or cover with circular holes in its surface shown in FIG. 3. As a method for achieving the results intended with the instant BPS invention the granular diameter is designed to be in the average size of 1/64″ to 1/32″, so that the circular perforations possibly will be around a 1/16″ diameter hole size.

Furthermore, the approximate serum concentrations of the various minerals, electrolytes, and chemicals may be identified for the disclosed information (ref: NORMAL LABORATORY VALUES, MKSAP® 14).

TABLE 4

Bicarbonate: 23-28 meq/L (23-28 mmol/L)
Chloride: 95-105 mmol/L, 98-106 meq/L
Copper: 70-150 μg/DI
Creatinine: 0.8-1.3 mg/DI
Ferritin: 12-300 ng/Ml (men), 12-150 ng/Ml (women)
Glucose: 65-110 mg/DI
Inorganic phosphorous: 1-1.5 mmol/L
Ionized calcium: 1.03-1.23 mmol/L
Magnesium: 1.5-2 mEq/L
Phosphate: 0.8-1.5 mmol/L
Potassium: 3.5-5 mmol/L, 3.5-5.0 meq/L
Pyruvate: 300-900 μg/DI
Sodium: 135-145 mmol/L, 136-145 meq/L
Total calcium: 2-2.6 mmol/L (8.5-10.2 mg/DI)
Total serum iron: 65-180 μg/DI (men), 30-170 μg/DI (women)
Transferrin: 200-350 mg/DI
Zinc: 70-100 μmol/L

Out of the above electrolyte list, (Lab Values, Normal Adult: Laboratory Reference Ranges in Healthy Afults Page 1 of 6, https://emedicine.medscape.com/article/3282316-overview 8/5/2019), it is noted that the essential dry electrolyte salts of this system, which are a high priority for health and wellbeing, must include a granulated measured ratio mixture of electrolyte salts, in the correct biocompatible and bioidentical ratio: sodium, calcium, potassium, chloride, phosphate, magnesium, phosphorous, chromium, zinc, copper, iron, selenium, molybdenum, chloride, manganese, vanadium, and iodine which are all electrolytes. It is also a feature of this invention that the most nutritious compounds of the mineral, electrolyte, and chemical salts are chosen according to the latest food chemistry knowledge base. An example of advantageous compounding is choosing potassium ascorbate or potassium citrate over, or in addition to potassium chloride as a combination in order to add vitamin C to the mixture. The same option is advocated for magnesium citrate and calcium citrate substitution. It is possible to get these mineral salts from the foods we eat and the fluids we drink. However, due to increasing climate global warming with record-breaking high temperatures throughout the world, electrolyte depletion and chronic dehydration is more commonplace than ever before. To ensure the restoration of those electrolyte minerals to the human body under stress, depleted for any reason, the proper restorative BPS can be utilized instantly in a handheld sized saltshaker dispenser ideally with perforations at one end of approximately hole diameters of 1/16 inches. Medical professionals have reported a rapid recovery from heatstroke which can be a precursor to fainting and/or death. In the health literature, the deprivation of sufficient minerals in the diet, in a hot, dry summer environment with record-setting heatwaves is therefore a major universal health hazard. Such a bioidentical polysalt mixture as disclosed herein may be regarded as creating a proper prophylactic or therapeutic treatment of various deficiencies contained mixture of electrolytes to achieve a specific Biopolysalt combination which is biophilic and bioidentical. It can and should be adjusted for taste to match the familiar salty flavor of sodium chloride. However, to begin with, a starter 400 mg mix may consist of those listed with their percentage of the total in Table 5:

TABLE 5

| | | | |
|---|---|---|---|
| Sodium chloride | 70 | mg | 17.5% |
| Potassium chloride/phosphate/bicarbonate | 200 | mg | 50% |
| Calcium chloride/citrate | 50 | mg | 12.5% |
| Magnesium carbonate/hydroxide | 60 | mg | 15% |
| Zinc ascorbate | 15 | mg | 3.8% |
| Chromium Picolinate | 0.01 | mg | 0.003% |
| Chromium Nicotinate | 0.5 | mg | 0.13% |
| Manganese gluconate | 0.5 | mg | 0.13% |
| Selenium methionine | 0.017 | mg | 0.004% |
| Copper citrate | 1.0 | mg | 0.25% |
| Iodine (as kelp powder) | 0.5 | mg | 0.13% |
| Ferrous Aspartate | 2 | mg | 0.5% |
| Vanadium (vanadyl sulfate) | 0.5 | mg | 0.13% |
| Molybdenum (chelate) | 0.05 | mg | 0.013% |

The ideal route for the dietary absorption of nutrients and fluids is, of course, the digestive tract. However, if for any reason the digestive tract route cannot be used, parenteral administration of these substances is required to sustain life. Parenteral therapy is used for the administration of nutrients, special fluids, and/or electrolytes by injection. Examples of parenteral routes include intravenous injection (into veins) and subcutaneous injection (under the skin). Significant quantities of nutrient or electrolyte solutions that are injected subcutaneously must be isotonic with plasma or cellular damage will occur. Therefore, use of BPS while the oral and digestive tract is functional and available during a stressful event can avoid the necessity of medical intervention toward intravenous or subcutaneous injection.

1. A system for dispensing a Biopolysalt composition comprising:
a container that may be referred to as a saltshaker, wherein the saltshaker encloses a granulated measured ratio mixture of dry electrolytes, including but not limited to a potassium salt, sodium salt, calcium salt, magnesium salt, phosphorous, chromium, vanadium, zinc, copper, iron, iodine, selenium, molybdenum, chloride, manganese, bicarbonate, etc., where the ratio of potassium to sodium content is two to one, the ratio of calcium to magnesium is two to one, and the ratio of calcium to phosphorous is 1.6 to one, with a balanced amount of chloride to bicarbonate.

2. The system as in claim 1, wherein the granulated measured mixture of dry Biopolysalt electrolytes is adapted to be ground into an average granule size between 1/64 to 1/32 inches in diameter.

3. The system as in claim 2, wherein an additional enclosed ingredient includes the necessary, state-of-the-art anti-caking additive(s).

4. The system as in claim 3, wherein the container may be made from glass, plastic, metal, wood, or ceramic and furthermore, possess at one end, a snap-on or screw-on cap, termination, or cover with circular hole perforations of 1/16 inches diameter in its surface.

5. A saltshaker that encloses a Biopolysalt granulated mixture of electrolytes with grain size in the range of 1/64″ to 1/32″ diameter, which are bioidentical endogenous electrolytes found in the average human body.

6. The saltshaker of claim 5 wherein the saltshaker container may be made from glass, plastic, metal, wood, or ceramic and furthermore, possess at one end, a snap-on or screw-on cap, termination, or cover with circular hole perforations in its surface of approximately hole diameters of 1/16 inches.

7. The saltshaker of claim 6 wherein the Biopolysalt granulated mixture of electrolytes includes but is not limited to a potassium salt, sodium salt, calcium salt, magnesium salt, phosphorous, chromium, zinc, copper, iron, iodine, selenium, molybdenum, vanadium, chloride, manganese, bicarbonate, etc., with optimum compounding of but not limited to carbonate, bicarbonate, citrate, and chloride.

8. The saltshaker of claim 7 wherein the Biopolysalt granulated mixture of electrolytes further is where the ratio of potassium to sodium content is two to one, the ratio of calcium to magnesium is two to one, and the ratio of calcium to phosphorous is 1.6 to one, with a balanced amount of chloride to bicarbonate.

9. The saltshaker of claim 8 wherein an additional enclosed ingredient includes the necessary improved quality of the state-of-the-art anti-caking additive(s).

10. A method for regulating and creating a proper prophylactic or therapeutic treatment of various deficiencies contained mixture of electrolytes to achieve a specific Biopolysalt combination which is biophilic and bioidentical comprising the steps of:
a. providing a handheld sized container, jar, bottle, saltshaker enclosure;
b. causing circular perforations to be drilled, punched, or formed with resulting holes of 1/16 inch diameter in one end of said enclosure, where the one end may serve as a removable cap or cover which may be snap-on or screw-on style;
c. providing a granulated measured ratio mixture of dry electrolytes, including but not limited to a potassium salt, sodium salt, calcium salt, magnesium salt, phosphorous, chromium, zinc, copper, iron, iodine, selenium, molybdenum, vanadium, chloride, manganese, bicarbonate, etc., with optimum compounding of but not limited to carbonate, bicarbonate, citrate, chloride, where the ratio of potassium to sodium content is two to one, the ratio of calcium to magnesium is two to one, and the ratio of calcium to phosphorous is 1.6 to one, with a balanced amount of chloride to bicarbonate;
d. grinding, filtering, evaporating and/or using a sieve to achieve a grain size in the range of 1/64″ to 1/32″ diameter;
e. combining the mixture of the previous steps (c) and (d) into the enclosure provided in steps (a) and (b) to complete the proper, specific biopolysalt combination for tabletop use.
